(19) United States
(12) Patent Application Publication
(10) Pub. No.: US 2018/0071420 A1
(43) Pub. Date: Mar. 15, 2018

(54) Mouth guard cleaning and storage device and related methods

(71) Applicant: Beast Enterprises, LLC, La Jolla, CA (US)

(72) Inventor: Khris Bestel, Boise, ID (US)

(21) Appl. No.: 15/701,665

(22) Filed: Sep. 12, 2017

Related U.S. Application Data
(60) Provisional application No. 62/495,340, filed on Sep. 12, 2016.

(51) Int. Cl. A61L 2/26

(52) U.S. Cl. CPC A61L 2/26; A63B 71/085; A61L 2202/17

(57) ABSTRACT

Disclosed generally is a mouth guard cleaning and storage device. In use, the device may be used to clean and store mouth guards and other dental appliances. The more specific aspects of the disclosed device are described below with reference to the appended figures.

### CROSS-REFERENCE TO RELATED APPLICATIONS

This application claims the benefit and priority of U.S. Pat. App. Ser. No. 62/495,340 (filed Sep. 12, 2016) (conf. no. 3428) entitled “mouth guard/night guard and various dental appliance sanitary cleaning and storage system.” This provisional application is 11

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is generally in the field of sport health and disease prevention. Specifically, the disclosed subject matter is in the field of mouth guard or other dental appliance storage and maintenance.

### Background of the Invention

Mouth guards are protective coverings worn over teeth to protect teeth from injurious impact and grinding during sports or otherwise. After use, mouth guards are often rinsed and set aside until the next need for teeth and mouth protection arises. Studies have demonstrated that used mouth guards, even if rinsed, harbor large numbers of opportunistic and pathogenic bacteria, yeast, and mold. (See Richard T Glass, et. al., Protective Mouthguards: Do They Cause Harm?). Mouth guards are typically porous in nature and, as such, a mouth guard can become a reservoir for pathogenic organisms that cause gum disease and tooth decay. Thus, a need exits for a reliable cleaning system.

### SUMMARY OF THE INVENTION

In view of the foregoing, an object of this specification is to disclose a storage and hygienic cleansing receptacle. Another object of this specification is to disclose a device and related methodology that combats improper maintenance of mouth guards and correspondingly reduces the number and intensity of oral mucosal injury and disease.

In a typical embodiment, the device is defined by reservoir and an extraction platform with a drain grate that is fixated to a lid for the reservoir by a shaft. In practice, the extraction platform and lid may operate as a lift for non-contact insertion or extraction of a dental device from selected cleaning fluids contained within the reservoir. Suitably, the above described non-contact insertion/extraction may eliminate the concern of bacterial contamination to the cleansing fluid from human hand contact. In one embodiment, the drain grate features drainage openings which preferably allow for proper hands-free drainage of excess cleansing fluid from the dental device and permit immediate use of the device upon extraction.

In one embodiment, a mouth guard may be provided to the extraction platform and the platform lowered into the reservoir containing cleaning fluid. In one embodiment, the reservoir is defined by a cylindrical receptacle and an extraction platform. The extraction platform is a circular disk grating that is affixed to the distal end of a shaft on the lid. Suitably, the reservoir may exhibit male threading that mates with the female threading of the lid to enclose the dental device upon lowering of the extraction platform into the reservoir. A rubber cylindrical loop fixated to the lid prevents leaking of fluid when fastened to the internal threading of the receptacle wall. The compact design, leak-proof seal, and durable composition of the receptacle and lid make the device ideal for travel and suitable for numerous dental devices such as dentures, mouth guards, occlusal guards, invisalign aligners, and orthodontic retainers.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is a perspective view of a mouth guard cleaning and storage device in a closed configuration;

FIG. 2 is a perspective view of a mouth guard cleaning and storage device in an open configuration;

FIG. 3 is a bottom perspective view of a mouth guard cleaning and storage device.

FIG. 4 is an exploded view of a mouth guard and storage device in an open configuration;

FIG. 5 is an environmental view showing a mouth guard being extracted from a cleansing fluid by a mouth guard cleaning and storage device in an open configuration;

FIG. 6 is an environmental view showing a mouth guard being stored and cleaned by the mouth guard cleaning and storage device in a closed configuration; and,

FIG. 7 is a perspective view of an alternate embodiment of a mouth guard cleaning and storage device.

In the figures, the following reference numerals represent the associated components of the disclosed device and system:
mouth guard cleaning and storage device—1000;
   lid—1100;
      top surface—1110;
      side wall—1120;
         internal threading—1221;
         external crevices—1122;
      rubbing sealing loop—1130;
   shaft—1200;
      lid connect end 1210;
      horse shoe platform connector end 1220;
   extraction platform—1300;
      drainage grating—1310;
         drain hole—1311;
      extraction platform shaft port—1320;
      peripheral lip—1330;
   receptacle—1400;
      external threading 1410;
      fill-line —1420;
mouth guard—2000; and,
Cleaning solution—3000.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

Disclosed generally is a mouth guard cleaning and storage device. In use, the device may be used to clean and store mouth guards and other dental appliances. The more specific aspects of the disclosed device are described below with reference to the appended figures

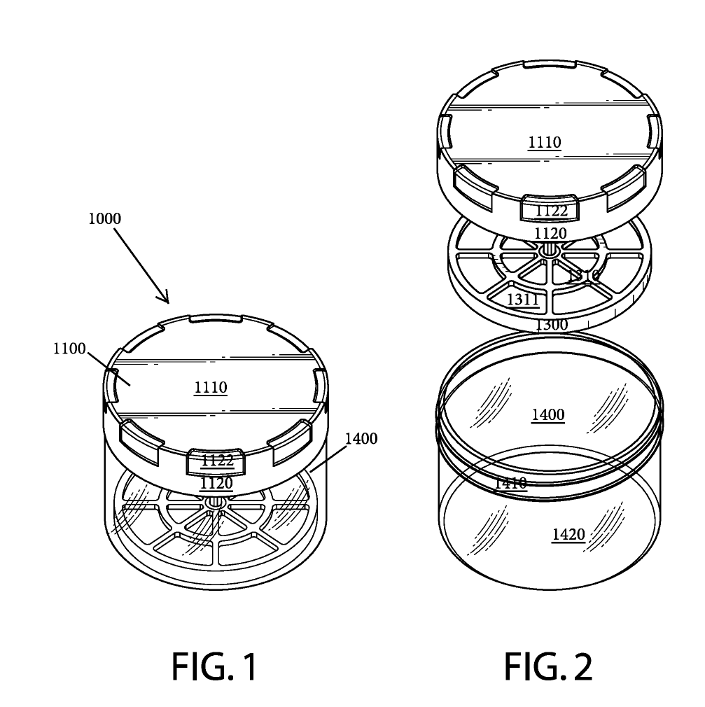

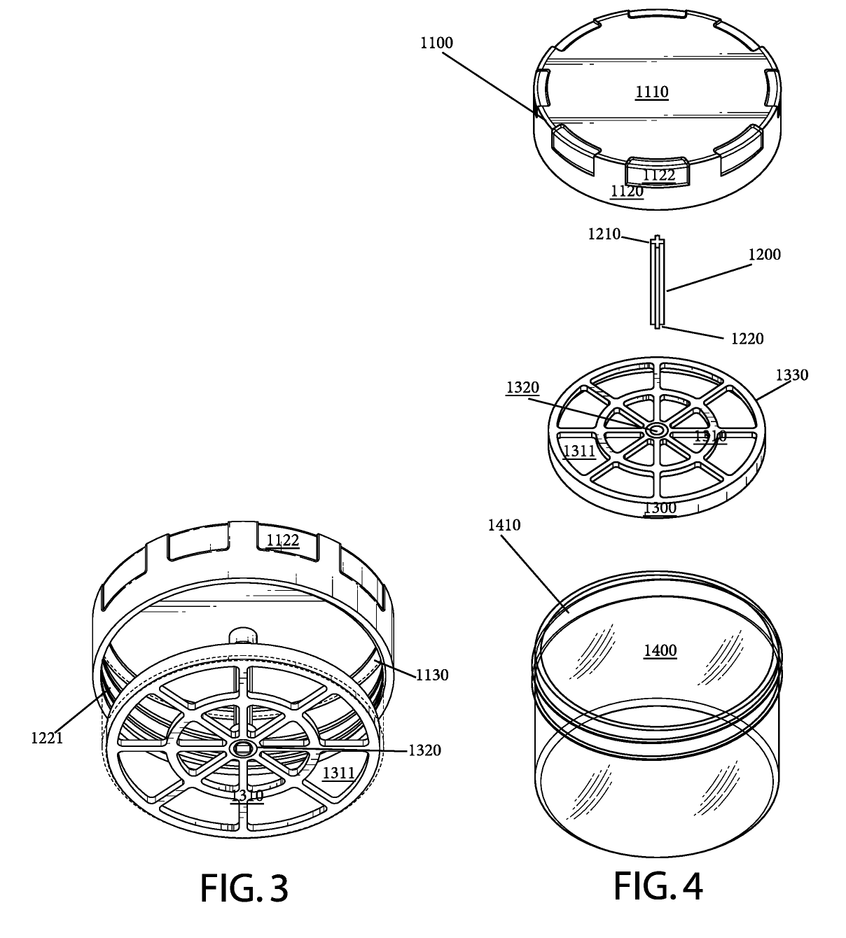

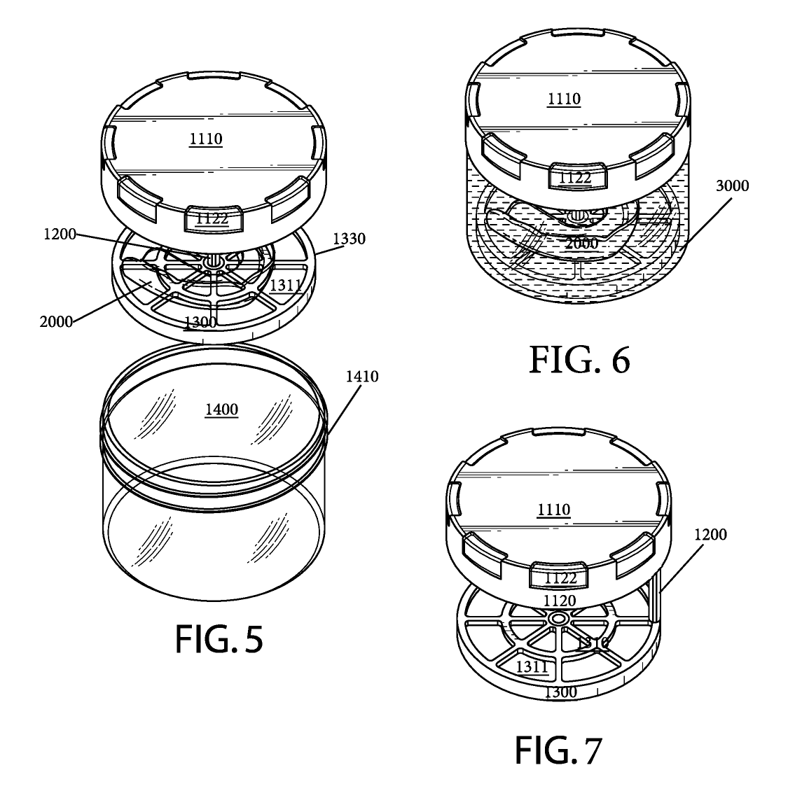

FIG. 1 is a perspective view of a mouth guard cleaning and storage device 1000 in a closed configuration. FIG. 2 is a perspective view of a mouth guard cleaning and storage device 1000 in an open configuration. FIG. 3 is a bottom perspective view of the mouth guard cleaning and storage device 1000 in a closed configuration. FIG. 4 is an exploded view of mouth guard cleaning and storage device 1000 in an open configuration. FIG. 5 is an environmental view showing a mouth guard being extracted from a cleansing fluid by a mouth guard cleaning and storage device 1000 in an open configuration. FIG. 6 is an environmental view of showing a mouth guard being stored and cleaned by the mouth guard cleaning and storage device 1000 in a closed configuration. As shown in the figures, the cleaning and storage device 1000 comprises a lid 1100, a shaft 1200, an extraction platform 1300, and a receptacle 1400.

FIGS. 1 through 4 show the lid 1100. Referring to those figures, the lid 1100 may be defined by a disk with a top surface 1110 and sidewall 1120. Suitably, the side wall may be defined by a hollow cylinder that projects coaxially from the top surface 1110. In one embodiment, the side wall features internal female type threading 1121 for, as discussed below, mating of the lid with the receptacle 1400. Additionally, the side wall exhibits external crevices 1122. Referring now to FIG. 3, a rubber sealing loop 1130 is fixated to the internal surface of the lid 1100 to create a leak-proof seal with the receptacle 1400. The lid also features a coaxial shaft port for, as discussed below, coaxially affixing the shaft 1200.

FIGS. 1 through 3 also show the shaft 1200. The shaft is an elongated member with a lid connector end 1210 and a horse shoe platform connector end 1220. As shown, the shaft 1200 coaxially fixates the lid 1100 to the extraction platform 1300. The shaft features a male type lid connector end 1210 for fixation to the lid 1100 shaft port, and a male type horse shoe platform connector end 1220 for fixation to the extraction platform shaft port 1320, discussed below.

Referring to FIGS. 1 through 5, the figures show the extraction platform 1300. As shown, the extraction platform 1300 is circular disk composed of a peripheral lip 1330 encompassing the drainage grating 1310. The drainage grating 1310 allows for the passage of cleansing fluids through drain holes 1311. The center of the extraction platform features the extraction platform shaft port 1320 for a coaxial connection to the lid 1100 via the shaft 1200.

Finally, FIGS. 1, 2, 4 and 5 show a receptacle 1400. The receptacle exhibits external threading 1410 for mating with the lid side wall internal threading 1221 and lid rubber sealing loop 1130. The receptacle wall external surface also features a printed fill-line 1420.

As shown in FIGS. 1 through 5, the lid 1100 is fastened to the extraction platform 1300 by the shaft 1200. The extraction platform 1300 and the shaft, 1200 are lowered into the receptacle 1400 with the lid mating with the receptacle external threading 1410 creating a seal between the lid 1110, the rubber sealing loop 1130, and the receptacle 1400. When in use, as shown in FIGS. 1 and 6 both the shaft 1200 and extraction platform 1300 are completely enclosed within the receptacle 1400.

FIG. 5 is an environmental view showing a mouth guard being extracted from a cleansing fluid by a mouth guard cleaning and storage device 1000 in an open configuration. FIG. 6 is an environmental view of showing a mouth guard being stored and cleaned by the mouth guard cleaning and storage device 1000 in a closed configuration. As shown in FIGS. 5 and 6, to initiate cleansing and storage, a mouth guard 2000 is placed on the extraction platform 1300 with the curvature of the device embracing the shaft 1200. A certain volume of cleaning solution 3000 is poured into the receptacle 1400 to meet the receptacle external fill-line 1420. The mouth guard 2000 and extraction platform 1300 are lowered into the receptacle 1400 via meeting the lid 1100 with the walls of the receptacle and submerged in the cleaning solution 3000.

Twisting the lid 1100 secures the lid to the receptacle 1400 through mating of the internal threading of the lid 1221 with the receptacle external threading 1410. FIG. 6 shows the cleansing process of the dental device in a closed configuration. For extraction and use, the lid 1100 is removed from the receptacle 1400, drawing the mouth guard 2000 stationed on the extraction platform 1400 out of the cleaning solution 3000 in a plunge-like manner. FIG. 5 shows the dental device being extracted from the receptacle. Excess solution 3000 is drained from the mouth guard 2000 and extraction platform via the drain holes 1311 of the drainage grating 1310 and collects back into the receptacle 1400.

FIG. 7 is a perspective view of a mouth guard cleaning and storage device 1000 in an alternate configuration. As shown in the figures, the cleaning and storage device 1000 comprises a lid 1100, a shaft 1200, an extraction platform 1300, and a receptacle 1400. Different from the initial embodiment, shown, the lid 1100 features an off-coaxial shaft port for, as discussed below, affixing the shaft 1200. As above, the shaft 1200 may be an elongated member with a lid connector end 1210 and a horse shoe platform connector end 1220. As shown, the shaft 1200 off-coaxially fixates the lid 1100 to the extraction platform 1300. As shown, the extraction platform 1300 is circular disk composed of a peripheral lip 1330 encompassing the drainage grating 1310. The drainage grating 1310 allows for the passage of cleansing fluids through drain holes 1311.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A cleaning and storage device.

2. The cleaning and storage device of claim 1 comprising:
a lid;
a shaft;
an extraction platform; and
a receptacle.

3. The cleaning and storage device of claim 1 where the lid further comprises:
a top surface;
a sealing rubber loop attached to the internal top surface;
a cylindrical side wall with external crevices; and
a cylindrical side wall with internal threading.

4. The cleaning and storage device of claim 1 where the shaft comprises:
a male type end for fixation to the lid; and
a male type horse shoe end for fixation to the extraction platform.

5. The cleaning and storage device of claim 1 where the extraction platform comprises:
a circular disk with a peripheral lip;
a circular disk composed of drainage grating;
a circular disk containing drain holes.

6. The cleaning and storage device of claim 1 where the receptacle comprises:
a cylindrical receptacle with side walls exhibiting external threading; and
a cylindrical receptacle featuring a printed fill-line on the external side wall.
